SEQUENCE LISTING (5 sequences)

SEQ ID NO: 1 - DNA, 65 bp, Artificial Sequence
  Synthetic Construct
gacgtcgcattcactgtcctgaacttcaattcccagcaacccaaccggccaactgcagccccttc

SEQ ID NO: 2 - DNA, 30 bp, Artificial Sequence
  Synthetic Construct
ggacgcgtcctcgaggtcagcttctagagg

SEQ ID NO: 3 - DNA, 34 bp, Artificial
  Synthetic Construct
aggcctgcaggaattccgtcgagttaattcccca

SEQ ID NO: 4 - DNA, 23 bp, Artificial
  Synthetic Construct
gggaagtcgcaaagttgtgagtt

SEQ ID NO: 5 - DNA, 17 bp, Artificial
  Synthetic Construct
cttcatggtgattttgg

(19) United States
(12) Patent Application Publication
(10) Pub. No.: US 2006/0058257 A1
(43) Pub. Date: Mar. 16, 2006

(54) Influencing angiogenesis using CD66a

(76) Inventors: Christoph Wagener, Hamburg (DE); Suleyman Ergun, Hamburg-Eppendorf (DE); Andrea K. Horst, Eppendorf (DE)

Correspondence Address: INTELLECTUAL PROPERTY / TECHNOLOGY LAW, PO BOX 14329, RESEARCH TRIANGLE PARK, NC, 27709, US

(21) Appl. No.: 11/224,162

(22) Filed: Sep. 12, 2005

Related U.S. Application Data
(63) Continuation-in-part of application No. 09/831,794, filed on Aug. 3, 2001, now abandoned.

(51) Int. Cl. A61K 48/00; A61K 39/395

(52) U.S. Cl. 514/44; 424/144.1

(57) ABSTRACT

The invention relates to a method of influencing angiogenesis by administering a of a compound in a pharmaceutically compatible carrier selected from the group consisting of (i) CD66a, a CD66a variant, CD66a fragments or CD66a-derived glycostructures, or CD66a ligands, or (ii) of anti-CD66a specific antibodies and anti-sense oligonucleotides.

### CROSS REFERENCE TO RELATED APPLICATIONS

The present application claims priority and is a Continuation-in-Part application of co-pending U.S. Application Ser. No. 09/831,794 with a filing date of Aug. 3, 2001, the contents of which are hereby incorporated by reference herein for all purposes.

### BACKGROUND OF THE INVENTION

1. Field of the Invention

The invention relates to a pharmaceutical composition for influencing angiogenesis. In one case, angiogenesis may be improved by administration of CD66a or substances initiating the formation of CD66a, while in the other case angiogenesis may be inhibited by using substances preventing interaction between CD66a and CD66a ligands.

2. Background of the Related Art

The formation of blood vessels (angiogenesis) is in many diseases an important step, which may contribute to curing, on the one hand, or shall desirably be prevented in other cases. Improving angiogenesis is very desirable e.g. for cardiovascular diseases to treat angina pectoris or heart attacks or cerebral infarctions, for example. On the other hand, the inhibition of the vascular supply of malignant solid tumors in humans and animals is a promising approach in tumor therapy. Angiogenesis inhibitors, such as endostatin, directly attack normal and thus genetically stable endothelial cells of the blood vessels supplying a tumor, cause them to die off and thus stop the supply of the tumor cells with nutrient-containing blood (cf. Kerbel, R., Nature, 390, p. 335 et seq., 1997). This leads to a regression of blood vessels and tumor mass. Since contrary to the tumor cells the endothelial cells are genetically stable, resistances do not form as is the case e.g. in a cytostatic therapy aiming directly at the tumor cells. By inhibiting angiogenesis the growth of human tumors could be blocked in experimental models. Some angiogenesis inhibitors are meanwhile tested clinically (cf. Hanahan et al., Cell 86, 353-364, (1996), Hurwitz et al. (2005). J. Clin. Oncol. 23, 3502-3508).

The supply of tissues with new vessels is a complex process in which a number of biomolecules are involved. Tumors produce soluble mediators, for example, which initiate the formation of new vessels. When angiogenesis proceeds, adhesion molecules play a central part. They control the communication of vessel cells with one another and with the surrounding connective tissue. Finally, various proteinases are also involved in the neovascularization.

### SUMMARY OF THE INVENTION

It is the object of the present invention to provide a possibility of improving or inhibiting angiogenesis as desired. In case angiogenesis is inhibited, a form of cancer therapy without the development of resistances shall thus be provided, i.e. in particular tumor-accompanying angiogenesis shall be influenced within the meaning of a reduction of angiogenesis.

According to the invention, this is achieved by the subject matters defined in the claims.

The subject matter of the present application is in particular a pharmaceutical composition suitable to regulate angiogenesis. Such a composition comprises:

(a) for positive regulation one or more bodies of CD66a, CD66a variants having angiogenic activity, CD66a fragments or CD66a-derived glycostructures, or CD66a ligands, ligand fragments or structures derived therefrom, as well as substances inducing the expression of CD66a or CD66a ligand, or

(b) for negative regulation one or more bodies of substances which inhibit the interaction between CD66a and CD66a ligands or substances which inhibit the expression of CD66a or CD66a ligand.

### BREIF DESCRIPTION OF THE FIGURES

FIG. 1 shows localization of CD66a in the vessels of a human Leydig cell tumor. The immunohistochemical staining was carried out using the 4D1/C2 antibody (a) One of the stained tumor capillaries is marked by an arrow (x350); (b) Enlargement of a region from FIG. 1a. The arrow points to the staining of an endothelial cell (x950).

FIG. 2 shows the chemotactic effect of CD66a (=BGP) on HDMEC.

FIG. 3 shows the proliferation of HDMEC following stimulation using CD66a (=BGP).

FIG. 4 shows the effect of CD66a on the formation of capillary-like vascular tubes in cell culture, (a) in the presence of the angiogenesis factor VEGF (50 ng/ml), capillary-like structures develop; (b) shows the result of an experiment in which the capillary formation was investigated in the presence of VEGF (50 ng/ml) and CD66a (150 ng/ml); (c) shows that capillary-like structures appear in the endothelial cells were cultured in the presence of CD66a (300 ng/ml) and in the absence of VEGF; (d) shows cappilaries fully inhibited in the endothelial cells that were cultured in the presence of the monoclonal 4D1/C2 antibody

FIG. 5 shows the generation of Tie2-CD66a-transgenic mice. (A) Transgenic construct for the generation of Tie2-CD66a transgenic mice. The Ceacam1 (CD66a)-L cDNA with an additional SV40 polyadenylation signal was cloned into the Sse63871 and Mlu I restriction sites, downstream of the Tie2-promoter and upstream of a Tie2-intronic enhancer. The arrow indicates the position of the probe used for genotyping transgenic mice by Southern blots and the fragment that was amplified by PCR for the identification of transgenic animals, respectively; (B) Southern blot of Tie2-CD66a (i.e. Ceacam1) transgenic founder lines. Microinjection of oocytes with the construct depicted in FIG. 5A yielded 5 founder lines (Tg). Experiments were performed with mice derived from 2 transgenic lines. WT: WT littermates; CT: control; (C) Flow cytometric analysis using anti-PEACAM-1 and anti-CEACAM1 (i.e. CD66a) double labeling of whole embryo (10.5 dpc) cell suspensions. WT embryos (WT, left panels) are compared to Tie2-Ceacam1 (i.e. CD66a) transgenic embryos (Tg, left panels. Experiments were performed with three embryos per line.

FIG. 6 shows the induction of colonic carcinomas by azoxymethane in Tie2-CD66a transgenic mice and wild type mice.

FIG. 7 shows the growth of HT-29 colonic carcinomas in SCID mice receiving either non-immune IgG (control), soluble recombinant murine CD66a (CC1) or anti-murine CD66a (CC1) antibody AgB10; CC1 is a synonym for CD66a.

### DETAILED DESCRIPTION OF THE INVENTION

The protein CD66a, which is also referred to as biliary glycoprotein (BGP), transmembrane carbon embryonic antigen or human C-CAM or CEACAM1 is a special adhesion molecule. The term CD66a is used below. The gene coding for CD66a has already been cloned (Hinoda et al., PNAS 85, 6959-6963, 1988). The applicants of the present application described in 1991 already the only CD66a-specific monoclonal antibody world-wide (Drzeniek et al., Cancer Letters 56, 173-179 (1991); Stoffel et al., J. Immunol. 150, 4978-4984 (1993)). This antibody is referred to as 4D1/C2 and was deposited with DSMZ (Deutsche Sammlung von Mikroorganismen und Zellkulturen [German-type Collection of Microorganisms and Cell Cultures], Mascheroder Weg, Braunschweig, under accession number DSM ACC2371 on Oct. 22, 1998.

It has now been found surprisingly that the CD66a factor is expressed in tumor capillaries whereas the blood vessels of the corresponding normal tissues are negative.

In a human Leydig cell tumor, the individual stages of the neovascularization could be traced accurately. In this connection, it was found that certain stages of neovascularization can be correlated with the occurrence of the following factors:

1. proliferation of endothelial cells: VEGF (vascular endothelial growth factor), VEGF receptors

2. formation of vascular lumens: CD66a

3. next differentiation step: endostatin

4. next differentiation step: angiostatin

In recent experiments conducted by the inventors using chicken embryos it could be shown that CD66a is a potent angiogenic factor and improves the neovascularization of normal and tumoral tissues.

It could also be shown that CD66a was blocked by an antibody directed against CD66a and the formation of capillaries necessary for tumor growth is inhibited. Tumor growth can no longer take place.

CD66a can be detected in human tumors in newly formed blood vessels (capillaries) in a defined differentiation window, namely in the stage of lumen formation. In an in vitro differentiation model a monoclonal CD66a antibody inhibits the formation of vascular tubes (tube formation) by human endothelial cells. These results prove that CD66a plays an essential part in angiogenesis. It follows from the expression of CD66a in tumoral vessels and the in vitro inhibition of capillary structure formation by a monoclonal CD66a antibody that tumor angiogenesis can be inhibited by functionally blocking CD66a.

Experiments conducted with transfectomas have shown that CD66a binds to itself (homeotypical binding) and to other members of the CD66 family. The localization of CD66a in newly formed endotheliums at the basal cell pole and the inhibition of capillary formation indicate that CD66a interacts with components of the extracellular matrix.

Antibodies, peptides, proteins or other agents which bind specifically to one or more functional domains of CD66a or its ligands are particularly suitable for the CD66a inhibition desired according to the invention in one respect. Monoclonal antibodies which are directed against adhesive, functionally significant domains of CD66a are preferably used. Preferably, the antibodies are directed against the B1 domain of CD66a, in particular against the carboxy terminal end of the B1 domain of the sequence published by Hinoda et al. (1988). Furthermore, CD66a has glycostructures which may have an angiogenic effect, e.g. LewisX and sialyl-LewisX groups. The above-mentioned monoclonal CD66a antibody 4D1/C2 is preferably used. This leads to an inhibition of tumor angiogenesis via a functional inactivation of CD66a. CD66a is in this connection functionally inactivated by inhibiting the interaction between CD66a and possible ligands. Here, structures that mediate interaction are blocked. Furthermore, soluble ligands or soluble ligand domains may also be used to block the interaction. The invention also relates to the use of recombinant domains that correspond to a CD66a fragment and to fragments of antibodies which react substantially with the epitope of CD66a. As a result, the signal chain starting from CD66a is blocked. The employed compounds may also be modified suitably to bind e.g. irreversibly to the receptor.

In particular, CD66a as a whole molecule, CD66 variants having angiogenic activity, CD66a domains as well as specific glycostructures of CD66a are suitable for the angiogenesis improvement desired according to the invention in one respect. In these cases, the soluble molecule form is applied to the sites of the body where angiogenesis shall be triggered (e.g. in the cardiac muscle). A DNA may also be used which codes for CD66a or parts of the CD66a protein. The DNA may also be integrated in vectors that are common in gene therapy (e.g. adenoviruses). Synthesis of the protein may also be achieved by administration of simple plasmid DNA. The positive influence of angiogenesis is affected by improving interactions between CD66a and CD96a ligands.

Methods of obtaining the above-mentioned antibodies which may be used for inhibiting angiogenesis are known to a person skilled in the art and comprise e.g. as to polyclonal antibodies the use of CD66a or a fragment thereof as immunogen for immunizing suitable animals and obtaining serum. The person skilled in the art is also familiar with methods of producing monoclonal antibodies. For this purpose, e.g. cell hybrids are produced from antibody-producing cells and bone marrow tumor cells (myeloma cells) and cloned. Thereafter, a clone is selected which produces an antibody specific to CD66a. This antibody is then produced according to standard methods. Examples of cells that produce antibodies are spleen cells, lymph node cells, B lymphocytes, etc. Examples of animals that may be immunized for this purpose are mice, rats, horses, goats and rabbits. The myeloma cells may be obtained from mice, rats, humans or other sources. The cell fusion may be carried out e.g. by the generally known method of Köhler and Milstein. The hybridomas obtained by cell fusion are screened using CD66a according to the enzyme-antibody method or according to a similar method. Clones are obtained e.g. with the boundary dilution method. The resulting clones are implanted intraperitoneally into BALB/c mice. Ascites is removed from the mouse after 10 to 14 days, and the monoclonal antibody is purified by known methods (e.g. ammonium sulfate fractionation, PEG fractionation, ion exchange chromatography, gel chromatography or affinity chromatography). The collected antibody may be used directly or a fragment thereof may be employed. In this connection, the term “fragment” means all parts of the antibody (e.g. Fab, Fv or single chain Fv fragments) which have an epitope specificity the same as that of the complete antibody.

In one embodiment, said monoclonal antibody is an antibody originating from an animal (e.g. mouse), a humanized antibody, a chimeric antibody or a fragment thereof. Chimeric antibodies which are similar to human antibodies or humanized antibodies have a reduced potential antigenicity but their affinity over the target is not lowered. The production of chimeric and humanized antibodies or of antibodies similar to human antibodies was discussed in detail (Noguchi, Nippon Rinsho, 1997, 55(6) pp. 1543-1556; van Hogezand, Scand. J. Gastroenterol. Suppl., 1997, 223, pp. 105-107). Humanized immunoglobulins have variable framework regions which originate substantially from a human immunoglobulin (designated acceptor immunoglobulin) and the complementarity of the determining regions which originate substantially from non-human immunoglobulin (e.g. from mouse) (designated donor immunoglobulin). The constant region(s) originate(s), if available, also substantially from a human immunoglobulin. When administered to human patients, humanized (and human) anti-CD66a antibodies according to the invention offer a number of advantages over antibodies from mice or other species: (a) the human immune system should not regard the framework or the constant region of the humanized antibody as foreign and therefore the antibody response to such an injected antibody should be less than that to a fully foreign mouse antibody or a partially foreign chimeric antibody; (b) since the effector region of the humanized antibody is human it might interact in a better way with other parts of the human immune system, and (c) injected humanized antibodies have a half life substantially equivalent to that of naturally occurring human antibodies, which permits administering smaller and less frequent doses as compared to antibodies of other species.

The above-described conventional technology may also be supplemented or replaced using recombinant phage libraries (Felici et al., Biotechnol. Rev. 1, pp. 149-183 (1995); Hoogenboom et al., Immunotechnology 4, pp. 1-20 (1998)). Recombinant phage libraries may have random peptide structures in the antigen-binding regions of the phage-presented antibody fragments. The advantage of this technology is inter alia that in cloned phages the information on the amino acid sequence of the antigen binding structures is directly available.

The domains of CD66a or the CD66a ligands, whose blocking effects a functional inactivation of CD66a, may be recombined in any way and be used while being introduced into molecules which are suitable for therapeutic purposes (e.g. to achieve better immunological compatibility). The reactive domains may also be expressed according to molecular-biological standard methods, e.g. bacterially or in insect cells.

Interaction between CD66a and potential ligands may preferably be inhibited in the following ways (negative regulation):

inhibition by antibodies and antibody fragments against the functional domain of CD66a,

inhibition by antibodies and antibody fragments against the functional domains of the CD66a ligands,

inhibition by the functional domain of CD66a,

inhibition by the functional domain of the CD66a ligands,

inhibition of the endogenous formation of CD66a or CD66a ligands using anti-sense oligonucleotides.

Interaction between CD66a and potential ligands may preferably be improved in the following ways (positive regulation):

application of the native molecule purified by means of biochemical methods,

application of recombinant CD66a fragments,

application of CD66a variants having angiogenic activity

application of angiogenicly active glycostructures isolated from CD66a,

application of glycostructures prepared in a fully synthetic or partially synthetic way, whose structure was derived from angiogenicly active glycostructures of CD66a,

application of a DNA, which codes for the complete CD66a protein thereof, in the form of suitable vectors or plasmids,

application of a DNA, which codes for isoforms or fragments of CD66a, in the form of suitable vectors or plasmids.

The pharmaceutical compositions according to the invention may be administered in any way suitable to reach the desired tissue. The administration is preferably carried out parenterally, particularly orally, intravenously or intratumorally. For the purpose of administration, the substance is used in a formulation suitable for the respective kind of administration using corresponding common pharmaceutical excipients. Orally applicable pharmacons are developed in two ways. On the one hand, interaction between ligand and receptor may be modeled e.g. by X-ray structural analysis or NMR spectroscopy. On the other hand, chemical combinatorial libraries (Myers, Current Opinion in Biotechnology 8, pp. 701-717 (1997) may be used. Here, the interaction of the ligand or receptor is examined with initially largely randomly combined chemical compounds. If binding was detected, the binding properties can be defined in more detail by selecting similar compounds.

Dosage and posology of the administration of the compounds according to the invention are determined by a physician on the basis of the patient-specific parameters, such as age, weight, sex, severity of the disease, etc. For example, effective dosages may vary from about 0.01 mg/kg body weight to 200 mg/kg bodyweight, typically from about 0.1 mg/kg bodyweight to 10 mg/kg bodyweight depending on the administered compound and mode of administration.

According to the kind of administration, the medicament is formulated suitably, e.g. in the form of solutions, suspensions, as a powder, tablet or capsule or injection preparations which are produced according to common galenic methods.

The infusion or injection solutions are preferably aqueous solutions or suspensions, it being possible to produce them prior to use, e.g. from lyophilized preparations which contain the active substance as such or together with a carrier, such as mannitol, lactose, glucose, albumin or the like. The ready-to-use solutions are sterilized and optionally mixed with auxiliary agents, e.g. with preservatives, stabilizers, emulsifiers, solubilizers, buffers and/or salts for regulating the osmotic pressure. The sterilization may be obtained by sterile filtration through filters having a small pore size, whereupon the composition may optionally be lyophilized. Antibiotics may also be added to help maintaining sterility.

The pharmaceutical compositions contain a therapeutically active amount of one or more of the above-mentioned active substances together with common auxiliary agents and carrier substances. They are preferably organic or inorganic liquid pharmaceutically compatible carriers which are suitable for the desired administration and which do not interact negatively with the active components.

The pharmaceutical preparations according to the invention are sold as unit dosage forms, e.g. as ampoules.

The invention also relates to a method of producing a pharmaceutical composition, which is characterized in that the compound according to the invention is mixed with a pharmaceutically compatible carrier.

“Substances inhibiting the expression of CD66a or CD66a ligand” are administered preferably by means of gene therapy introducing into tumor cells e.g. anti-sense oligonucleotides to CD66a and/or CD66a ligand. These oligonucleotides are derived from the known sequences for CD66a or CD66a ligand (Hinoda et al., Proc. Natl. Acad. Sci. U.S.A. 85, p. 6959 (1988)). The anti-sense oligonucleotides may also reach the size of a DNA which is complementary to regions of the gene mRNA and binds thereto. Then, a duplex molecule forms which is taken away from the translation of the mRNA. Inhibition of the gene expression can be achieved in this way. The term “anti-sense oligonucleotide” comprises any DNA or RNA molecule which is complementary to regions of the CD66a RNA or CD66a ligand RNA, in particular mRNA and most particularly regulatory elements thereof, and effects inhibition of the gene expression by binding to these regions. In a particular embodiment of the invention, the anti-sense oligonucleotide is a CD66a specific small interfering RNA (siRNA) for silencing the CD66a gene (Kilic et al.: J. Biol. Chem. (2005), vol. 280, pp. 2361-2369). The anti-sense oligonucleotides may be available as such or, if they are relatively long, in the form of a vector or vector construct coding for them, which is sometimes also referred to as “minigene.” Such a vector may be a common expression vector. It may be favorable for the expression of the sequence coding for them to be controlled by a constitutive or inducible promoter, such as a tissue-specific or tumor-specific promoter. The anti-sense molecules may be introduced by common methods. If the anti-sense oligonucleotides are available as such or in the form of a vector coding for them, e.g. transfection techniques or packaging in liposomes is suitable.

“Substances which induce the expression of CD66a or CD66a ligand” are e.g. DNA molecules that code for CD66a or angiogenicly active CD66a fragments or for CD66a ligands or angiogenicly active ligand fragments or substances that activate a promoter, which is functionally linked with the CD66a gene. The expression is controlled by suitable regulatory sequences. The DNA is administered according to protocols known to a person skilled in the field of gene therapy. Thus, e.g. packaging of the DNA in viral particles (e.g. adenoviruses) or the administration of naked plasmid DNA is in consideration.

“CD66a variants” according to the invention refer to modified CD66a proteins, wherein the modifications are according to common methods known in the art. The modifications comprise substitutions, insertions or deletions of amino acids, which modify the structure of the protein, its angiogenic activity being substantially maintained. The substitutions comprise particularly “conservative” substitution of amino acid residues, i.e. substitutions for biologically similar residues, e.g. the substitution of a hydrophobic residue (isoleucine, valine, leucine, methionine, for example) for another hydrophobic residue, or the substitution of a polar residue for another polar residue (e.g. arginine for lysine, glutamic acid for aspartic acid, etc.). Deletions may result in the production of molecules markedly reduced in size, i.e. fragments which lack the transmembrane domain, for example. In this connection, the variants have an identity of at least 70%, preferably 80%, more preferably 90%, most preferably 95, 96, 97, 98 or 99%, with the amino acid sequence derived from the nucleotide sequence published by Hinoda et al. (1988).

According to the invention, the growth of all solid tumors of the body may be inhibited with the angiogenesis-inhibiting pharmaceutical composition. Examples are epithelial tumors (e.g. squamous epithelium, columnar epithelium, glandular epithelium, transitional epithelium), mesenchymal tumors (e.g. fibers, muscles, cartilages, and bone tissues), mixed tumors (mixed epithelial, mixed mesenchymal, epithelial-mesenchymal), tumors of the hematopoietic and lymphatic tissues (bone marrow, lymphatic tissue), tumors of the serous cavities (e.g. pulmonary pleura, heart sac, abdominal membrane, synovial membrane), tumors of the nervous system (e.g. ganglion cells, neuroepithelium, neroglia, meninges, sympathicus, peripheral nerves), tumors of the gastro-intestinal tract and tumors of individual organs. The growth of tumors of the bronchi and the lungs, breast, liver, bile, pancreas, kidneys and urinary tracts, stomach, large intestine, straight intestine, prostate and uterus are preferred according to the invention.

According to the invention, the neovascularization may be induced by the angiogenesis-improving pharmaceutical composition in diseases in which the disease-dependent occlusion of vessels results in an insufficient supply of the tissue with oxygen and nutrients. Cardiac diseases or insufficient blood supplies of the extremities in diabetics, heavy smokers or patients suffering from hypertension are to be mentioned as examples.

The invention is explained in more detail by means of the following examples.

### EXAMPLE 1

### Localization of CD66a in Tumor Capillaries

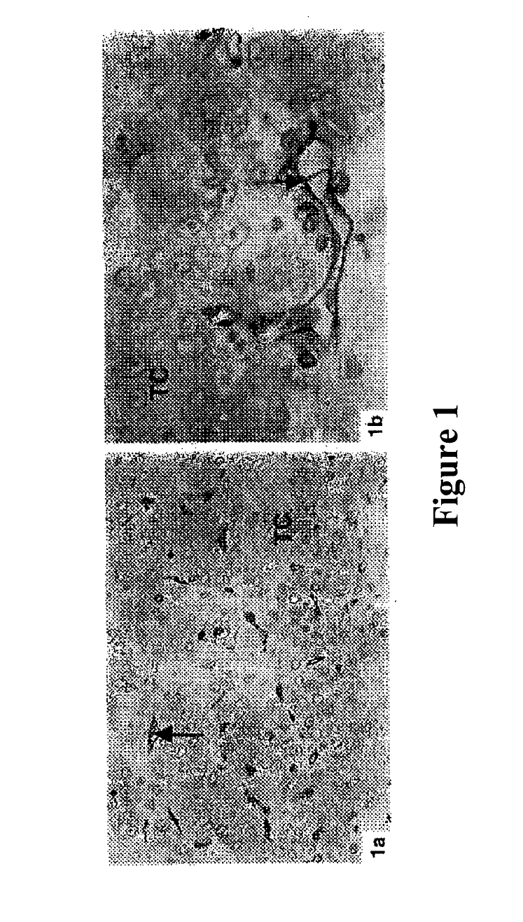

Tumors were stained immunohistochemically using the monoclonal anti-CD66a antibody 4D1/C2 and investigated by means of an optical microscope. For this purpose, an intensifying method using nickel and glucose oxidase was used in addition to the previously employed immunohistochemical methods (Prall et al. (1996), J. Histochem. Cytochem. 44, 35-41). Furthermore, electron-microscopic analyses were carried out following immunohistochemical staining using the monoclonal 4D1/C2 antibody (see FIG. 1).

Human testicular tumors, brain tumors as well as prostate, bladder and kidney carcinomas were examined immunohistochemically. CD66a was localized in endothelial cells and in the basal membrane of the tumor capillaries. Mature, non-proliferating resting vessels of the examined organs were negative. In case the tumor is divided into different zones in accordance with functional aspects, namely tumor cells, tumor margin and tumor environment, the positive immune response can be found in the newly formed tumor capillaries on the tumor margin. This indicates a function of CD66a in very early stages of neovascularization (neoangiogenesis).

### EXAMPLE 2

### Effect of CD66a on the Proliferation and Chemotaxis of Cultured Endothelial Cells

In order to test the effect of CD66a on the proliferation and chemotaxis of cultured endothelial cells, the glycoprotein was purified from membrane fractions of human granulocytes. The membrane fraction was isolated in accordance with established methods (Drzeniek et al. (1991), Cancer Letters 56, 173-179; Stoffel et al. (1993), J. Immunol. 150, 4978-4984). After extracting the membrane glycoproteins with a non-ionic detergent, they were bound to an immobilized monoclonal CD66 antibody and eluted using glycin-HCl at pH 2.2. Following neutralization the eluate was further separated by means of gel chromatography on Superdex 200 (Pharmacia). The CD66a-positive fractions were pooled. Contaminations in the low-molecular region were separated by means of ultrafiltration using a filter having an exclusion of 100 kD. In combination with a Western blot it was shown by means of SDS-PAGE in silver gel that the supernatant exclusively contained CD66a. This fraction was used for cell culture experiments with endothelial cells.

The experiments were carried out with two different human endothelial cell forms, namely with HUVEC (human umbelical vein endothelial cells) and HDMEC (human dermal microvascular endothelial cells).

The effect of CD66a on the proliferation was checked in a monolayer culture. Endothelial cells were seeded in a defined number on a microtitration plate. After 72 hours, the number of endothelial cells in stimulated and non-stimulated cultures was compared. It turned out that CD66a stimulated the proliferation of both cell lines in dose-dependent manner.

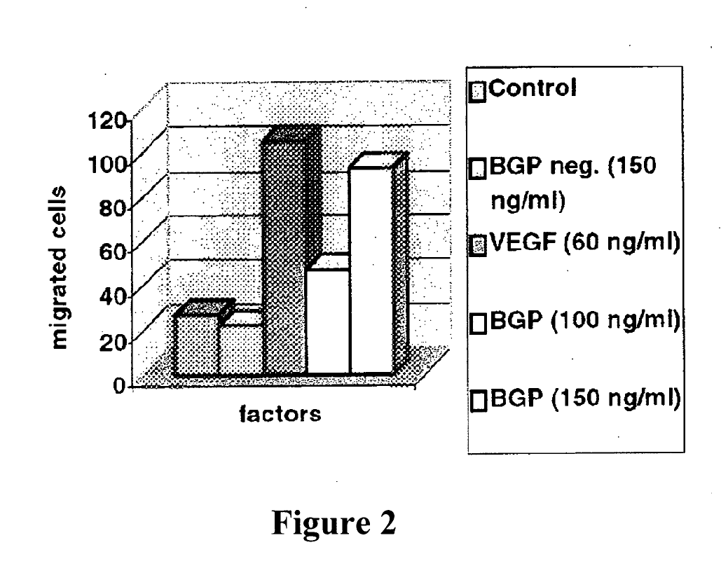

The effect of CD66a on chemotaxis was investigated in a two-chamber culture system (what is called a Boyden chamber). The cells are cultured in the top chamber. The bottom chamber contains chemotactic substances. Both chambers are separated by a polycarbonate filter permitting passage of the cells. After adding CD66a to the bottom chamber, a dose-dependent chemotactic effect showed on both endothelial cell lines. The effect of CD66a could be compared with the effect of VEGF. As evident from FIG. 2, CD66a (=BGP) has a chemotactic effect from a concentration of 100 ng/ml. With a concentration of 150 ng/ml the chemotactic effect is only slightly less than that of VEGF (vascular endothelial growth factor).

The chemotactic effect of CD66a was also analyzed in combination with VEGF and bFGF (basic fibroblast growth factor). The chemotactic effect of VEGF or bFGF was increased by CD66a by about 30% each.

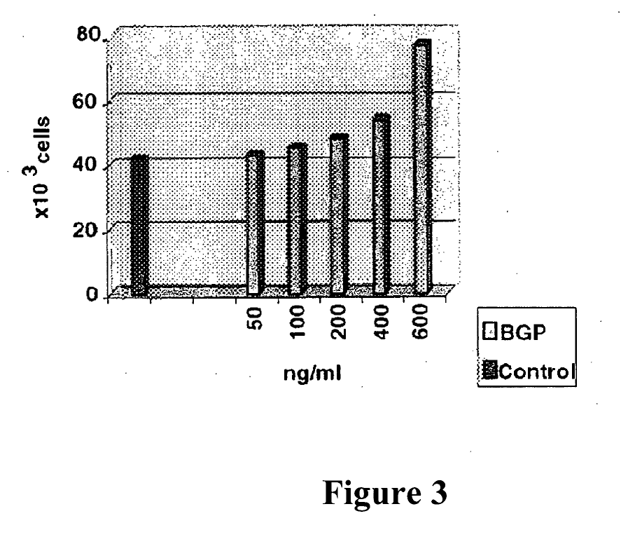

Cultured human microvascular dermatofibroblasts were incubated with CD66a (=BGP) in concentrations of 50, 100, 200, 400 and 600 ng/ml. From a concentration of 200 ng/ml a proliferation-increasing effect of CD66a could be detected. This is shown in FIG. 3.

Due to the positive effect on proliferation and chemotaxis CD66a fulfills the main criteria of angiogenesis factors.

### EXAMPLE 3

### Effect of CD66a on the Formation of Capillary-Like Vascular Tubes in Cell Culture

The test results described in Example 2 suggest that CD66a is causally involved in the formation of new vessels (neoangiogenesis). In order to check this hypothesis, animal experiments would be most suitable. However, since CD66a is a human glycoprotein, it has to be expected that due to the differences in the species the effect in the experimental animal shows no or only slight expression. The finding that the monoclonal anti-CD66a 4D1/C2 antibody shows good reaction in human tissues supports this assumption. The reaction is weak in the corresponding tissues of rats and mice and can be distinguished only with difficulty from a non-specific background reaction. The 4D1/C2 antibody obviously binds to an antigenic structure, which does not occur in rodents in this form.

In order to circumvent the problems caused by the differences regarding the species, cell culture models are used in which endothelial cells grow under certain conditions into vascular tubes which correspond to newly formed capillaries (tube formation). For this purpose, the cells are cultured in the presence of specific growth factors such as VEGF (vascular endothelial growth factor) or FGF-2 (fibroblast growth factor) in a connective tissue matrix. This culture form represents a good approach to in vivo conditions.

In order to investigate the significance of CD66a for the formation of capillaries, HUVEC and HDMEC cells were cultured in three-dimensional collagen I gels. In the presence of growth factors such as VEGF and FGF-2, the endothelial cells form tubular structures that correspond to newly formed capillaries. In the presence of the monoclonal CD66a 4D1/C2 antibody, the formation of vascular tubes was inhibited. Another monoclonal antibody T84.1, which is directed against the carcinoembryonic antigen (CEA) and crossreacts with CD66a (Drzeniek et al. (1991)), had no effect on the formation of tubes. These experiments prove a functional correlation between the expression of CD66a and the neoformation of capillary-like vascular tubes. The functional domain of CD66a is also defined by means of the antibody.

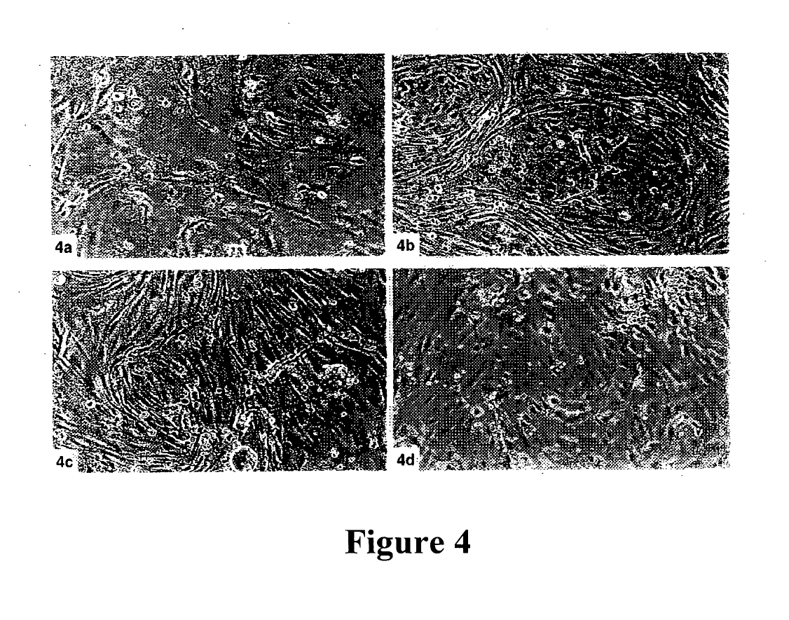

The results of the above experiments are shown in FIG. 4:

In the presence of the angiogenesis factor VEGF (50 ng/ml), capillary-like structures develop (see FIG. 4a). Capillary-like structures manifest themselves by way of tubes in which the longitudinal endothelial cells are arranged parallel. These tubes can be compared to fish schools. In the middle of FIG. 4a there is a region in which the endothelial cells are rounded. They are no tubes.

FIG. 4b shows the result of an experiment in which the capillary formation was investigated in the presence of VEGF (50 ng/ml) and CD66a (150 ng/ml). As compared to FIG. 4a, almost all endothelial cells are involved in the formation of tubes. Furthermore, a branching pattern can be seen which supports the further differentiation of the angiogenesis process. CD66a thus intensifies the angiogenic effect of VEGF.

In FIG. 4c, the endothelial cells were cultured in the presence of CD66a (300 ng/ml) and in the absence of VEGF. Capillary-like structures appear.

FIG. 4d shows the result of an experiment in which the endothelial cells were cultured in the presence of the monoclonal 4D1/C2 antibody. The formation of capillaries is fully inhibited. It follows from this experiment that the 4D1/C2 antibody binds to a domain of CD66a, which is essential for the formation of capillaries.

### EXAMPLE 4

### Transgenic Mice Models

In order to show the angiogenic action of CD66a, a transgenic CD66a mouse model (Tie2-CD66a-transgenic mice) has been developed on an FVB/N background. In this model, the CD66a transgene is expressed under the control of an endothial-specific promoter (Tie2 promoter). This promoter ensures that the transgene is expressed specifically in the endothelia of blood vessels.

### Cell Lines

Stable CD66a-expressing transfectant cells were generated with the murine endothelial cell line SVEC4-10 (SV40 immortalized endothelial cells from maxillary lymph node; ATCC) by retroviral infection (Kunath et al. (1995) Oncogene 11, 2375-82). CD66a-4L and CD66a-4L mutants (Y488F and Y488F;S503A) were generated by site-directed mutagenesis (Huber et al. (1999) J Biol Chem 274, 335-44). The Y488F;S503A mutant was generated by overlap PCR using primers AH1 (5′GAC GTC GCA TTC act GTC CTG AAC TTC AAT TCC CAG CAA CCC AAC CGG CCA ACT GCA GCC CCT TC3′) (SEQ ID NO: 1) and NBIT2 (McCuaig et al. (1993) Gene 127, 173-83). All cDNAs were cloned into pLXSN vector (Stratagene) for recombinant retrovirus production. Infected SVEC4-10 cell populations were selected with G418 (1 mg/ml, Invitrogen). Cell clones were derived by limited dilution. Before the transgene was introduced into oocytes, the effect of the gene construct was studied in in vitro cell cultures of murine endothelial cells. It was shown that the transgene changed the morphology of the cells towards a capillary-like structure.

### Generation of Tie2-CD66a- and Mutant Tie2-CD66a-Transgenic Mice

The transgenic construct pHHNS was obtained from Dr. Thomas N. Sato (Schlaeger et al. (1997) Proc Natl Acad Sci USA 94, 3058-63; Sato, T. N. http://cbi.swmed.edu/ryburn/sato). Unique restriction sites for Sse8387 I and Mlu I were added to the CD66a-4L cDNA by PCR, using the appropriate primers (AKH1: 5′GGA CGC GTC CTC GAG GTC AGC TTC TAG AGG3′ (SEQ ID NO: 2) and RAKH1: 5′AGG CCT GCA GGA ATT CCG TCG AGT TAA TTC CCC A3′) (SEQ ID NO: 3). The β-galactosidase (lacZ) cassette of the pHHNS vector was exchanged for the murine CD66a-4L cDNA (Kunath et al. (1995)) followed by a SV40 polyadenylation signal. The same basic construct was used to generate the mutant Tie2-CD66a-transgenic (DN-Tie2-CD66a-transgenic) vector, containing the Y488F;S503A mutant form of CD66a-4L instead of the WT (wild-type) cDNA. All modifications in the construct were subjected to DNA sequencing prior to use. Transgenic mice were generated by microinjection into FVB/N mouse oocytes of a Sal I fragment encompassing the Tie2 promoter and enhancer, the CD66a-4L cDNA and the SV40 polyadenylation signal. FBV/N mice were obtained from Jackson Laboratories. Five and three original founder lines were obtained, respectively, and mice derived from the F2 or subsequent generations were used in the experiments. Care of the mice was taken according to standards defined by the Canadian Council on Animal Care and Paragraph 8 of the German Law for the Protection of Animals.

### CD66a-Null Mice

CD66a-null mice were generated on C57B1/6 background by N. Beauchemin as described (Hemmila et al. (2004) J Virol 78, 10156-65).

### Genotyping of Mice

CD66a-null mice were genotyped by PCR (Hemmila et al. (2004)). The Tie2-CD66a-transgenic and DN-Tie2-CD66a-transgenic mice were characterized by Southern blotting after EcoRI restriction digest of genomic DNA from tail biopsies and by PCR. In both cases, an overlapping fragment of the Tie2-promoter and the N-terminal CD66a-domain was used as a probe or as a target for amplification by PCR, using primers 2T5 (5′GGG AAG TCG CAA AGT TGT GAG TT-3′) (SEQ ID NO: 4) and 46N1 (5′CTT CAT GGT GAT TTT GG-3′) (SEQ ID NO: 5).

### Flow Cytometric Analyses

CD66a-expression on the surface of SVEC4-10 transfected cells was analyzed as in Kunath et al. (1995). For mouse lung endothelial cell preparations from Tie2-CD66a- and DN-Tie2-CD66a-transgenic mice, CD66a and PECAM-1 (platelet-endothelial cell adhesion molecule) staining was performed as previously described (Nicosia & Ottinetti (1990) Lab Invest 63, 115-22). For CD66a, the monoclonal anti-CD66a-antibody AgB10 (Rudinskaya et al.: Biol. Membr. (USSR) 1987; (4):194-207 and Kuprina et al.; Histochemistry 1990; 94(2):179-86) was used.

### Ex vivo Aortic Ring Assays

Aortas were prepared from 8-10 week old Tie2-CD66a-transgenic, DN-Tie2-CD66a-transgenic FVB/N or CD66a-null mice, and their WT (wild-type) littermates. Aortic ring assays were performed as previously reported (Nicosia & Ottinetti (1990) Lab Invest 63, 115-22). For inhibition studies, anti-CD66a-antibody AgB10 was used (10 μg/mL). Endothelial cell outgrowth was monitored for 10 days. Statistics were performed starting at day 2. Pictures were taken with a Canon digital camera mounted on a Zeiss Axiovert microscope. For quantification, endothelial cell sprouts were counted and their total length was measured.

### In vivo Matrigel Plug Assays

Mice were injected subcutaneously with 0.5 ml of Matrigel (BD Biosciences) supplemented with either 200 ng of recombinant angiopoetin1, 120 ng of recombinant murine VEGF¹²⁰, or 120 ng of bovine bFGF (all from R&D Systems) as described (Passaniti, A. (1992) Lab Invest 67, 804; author reply 804-8). Controls did not contain any growth factors. Matrigel implants were removed after 14 or 21 days, and neo-vascularization was gauged after routine paraffin embedding by immunohistochemical analysis. To visualize blood vessels in the Matrigel plugs, FITC-labeled dextran (Sigma) was injected into the tail vein of the mice. After 5 minutes to allow sufficient circulation, mice were sacrificed. Subsequently, the plugs were removed and subjected to paraffin embedding.

### Immunhistochemical Analysis of CD66a- and PECAM-1-Expression

Immunohistochemical staining of CD66a and PECAM-1 was performed on paraffin embedded specimen as described (Jörns et al. (2003) Anat Embryol (Berl) 207, 85-94). A polyclonal anti-CD66a-antiserum (2456, 1:500) and an anti-PECAM-1 antibody (2 μg/ml, Biermann DPC) were used. CD66a- or PECAM-1-binding was visualized through alkaline phosphatase activity, using Avidin-Biotin-Complex-(ABC) Kits (Vector Laboratories) with either naphthol-AS-bisphosphate or Vector Blue™ (Vector Laboratories). Counterstaining of nuclei was performed with either Mayer's hemalaun (Merck) or NuclearFastRed. Sections were analyzed using a Zeiss Axioplan microscope.

### Statistical Procedures

Statistical analyses were carried out with Student's t-Test. P values<0.05 were considered to be statistically significant.

### EXAMPLE 5

### Endothelial Cell Outgrowth and Differentiation in Aortic Explants is Dependent of the Presence of CD66a and Can Be Abolished by anti-CD66a-Specific Antibodies

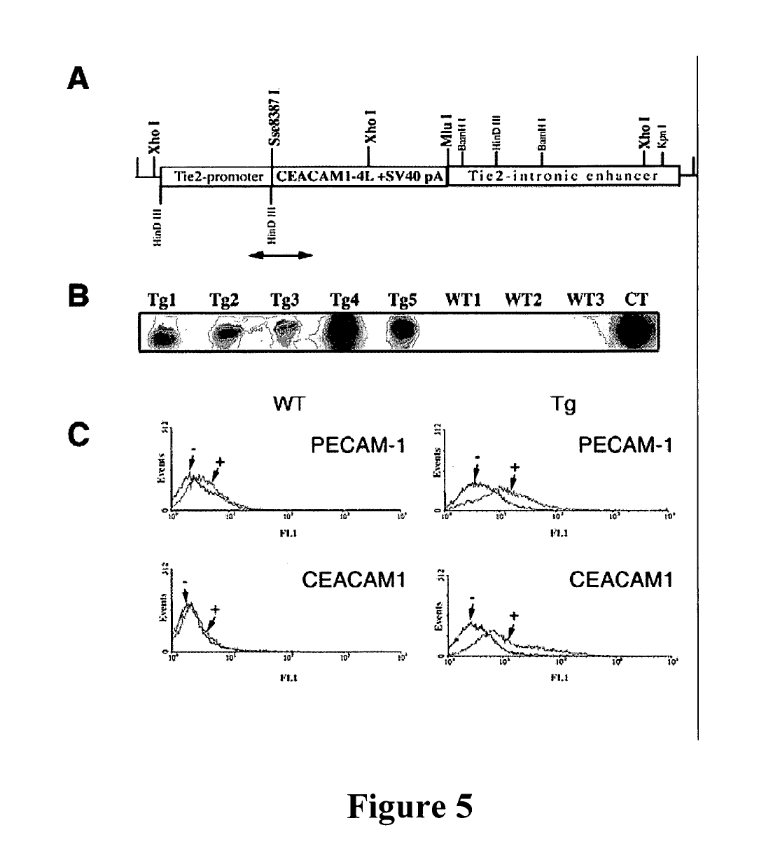

In order to define CD66a-mediated effects in early stages in angiogenesis in vitro and in vivo, three two mouse models were generated: in Tie2-CD66a-transgenic mice, WT CD66a-L was over-expressed in response to the endothelial cell-specific promoter control of the Tie2 receptor tyrosine kinase (FIGS. 5A and 5B). The same construct was used to obtain DN-(dominant-negative)Tie2-CD66a-transgenic mice over-expressing the Y488F;S503A-mutant form of CD66a-L in endothelia. This mutant was chosen as a DN form of CD66a, since its expression in murine SVEC impaired tube formation and cellular invasion capacities in both 2D and 3D experimental systems. Additionally, CD66a-deficient mice with systemic deletion of the CD66a-gene were used (Hemmila et al. (2004). CD66a over-expression in the endothelia of Tie2-CD66a- and DN-Tie2-CD66a-transgenic mice was verified in double-labeling procedures in flow cytometry (FIG. 5C) and Western Blotting. Macroscopically, no overt vascular damage or alterations were observed under physiological conditions in CD66a-null or Tie2-CD66a and DN-Tie2-CD66a -transgenic mice. In all Tie2-CD66a-transgenic mice and genetically unaltered animals, CD66a expression was found in a broad range of vessels, including small capillaries but also the endothelium of the aorta.

To compare the angiogenic properties of endothelial cells of CD66a-null and, Tie2-CD66a and DN-Tie2-CD66a-transgenic mice, aortic ring assays were performed (Nicosia & Ottinetti (1990)). Aortic endothelial cell invasion was more pronounced in Tie2-CD66a-transgenic mice compared to their WT siblings, though the complexity of the capillary network did not differ significantly between transgenic and WT animals. These results are also reflected in statistical analyses, with aortic endothelial tubes from Tie2-CD66a-transgenic mice exhibiting increased invasive sprouting and invasion capacities as opposed to their WT littermates.

Statistical analysis revealed that, in the DN-Tie2-CD66a-transgenic mice, capillary sprouts were significantly longer compared to the wild type littermates.

In contrast to this, in the DN-Tie2-CD66a-transgenic mice and the CD66a-null mice the structure of the endothelial network endothelial networks emanating from DN-Tie2-CD66a-transgenic mice exhibited reduced interconnections between individual endothelial protrusions. The aortic explants differed from the respective networks of the wild type animals. In the DN-Tie2-CD66a-transgenic mice endothelial cells remained isolated and were not incorporated into the network.

In a sharp contrast to these results, endothelial cell tubes protruding from aortic explants of CD66a-null mice formed a less complex endothelial network with poor endothelial tube extension compared to the dense network emerging from explants of their corresponding WT littermates. Additionally, buds were emerging at branching sites in the CD66a-null aortic explants that did not extend into sprouts or invade the ECM to interconnect with neighboring protrusions.

To further verify CD66a-dependent effects on endothelial cell outgrowth in the aortic explants, it was investigated whether anti-CD66a antibodies interfere with endothelial tube formation or network maintenance in the aortic explants. After application of the anti-CD66a-antibody AgB10 in micromolar concentrations to the growth media, newly-formed capillary networks of Tie2-CD66a-transgenic mice or WT animals were disrupted, and formation of isolated cell colonies was observed. Interestingly, the tubular structures emerging from the aortic explants of CD66a-null mice remained morphologically intact, whereas the network emerging from WT explants was affected. Appropriate isotype controls showed no effects on cellular endothelial tube structure. Since CD66a is a homophilic cell adhesion molecule, CD66a may mediate endothelial cell interactions in trans that may be disrupted by the application of anti-CD66a antibodies. Collectively, these results confirm that early stages of capillary network formation are a CD66a-dependent process.

### EXAMPLE 6

### Lack of Endothelial CD66a-Expression Provokes Impaired Neo-Vascularization and Severe Vascular Leakage in Vivo After Challenge With VEGF

To study the effects of CD66a-expression in endothelia or lack thereof in vivo on neo-vascularization, Matrigel plug assays in Tie2-CD66a-transgenic and CD66a-null mice were performed. Mice were injected Matrigel s.c. (subcutaneous), containing either Ang-1, bFGF or VEGF, and neo-vascularization was visualized after histological processing for immunoreactivity towards anti-CD66a- or anti-PECAM1-antibodies. VEGF-treated plugs retrieved from Tie2-CD66a-transgenic mice are vascularized extensively and show immunoreactivity towards anti-CD66a-antisera and an anti-PECAM-1-antibody, whereas endothelial cell populations in plugs retrieved from CD66a-null mice remained unstained. Strikingly, extravascular erythrocytes were detected in proximity of newly formed vessels in the plugs retrieved from CD66a-null mice. Tie2-CD66a transgenic mice exhibit higher neo-vascularization of the Matrigel implants upon angiogenic challenge with Ang-1, bFGF and VEGF, as compared to their WT littermates. In sharp contrast to these findings, the CD66a-null mice failed to vascularize the implant and only very few intact vessels were detected whereas the corresponding C57B1/6 WT mice responded to angiogenic stimuli by extended neo-vascularization of the Matrigel implants. Ang-1 and bFGF proved to be the most effective angiogenic stimuli. Based on the observations that CD66a-null mice exhibited vascular leakage in neo-vascularized areas of Matrigel implants and that the number of intact vessels was dramatically lower compared to their C57B1/6 WT littermates, the vessel integrity and stability might be altered in CD66a-null mice. In a sharp contrast, a considerable number of distinct small vessels were present in VEGF-treated implants retrieved from C57B1/6 WT mice. Exudation of FITC-labelled dextran was rather weak in the WT controls, implying a mild increase in vascular permeability only. No such dramatic effects were observed after treatment of the implants with Ang1 or bFGF, respectively. Without the addition of extra supplements to trigger neo-angiogenesis, the implants remained largely avascular.

### EXAMPLE 7

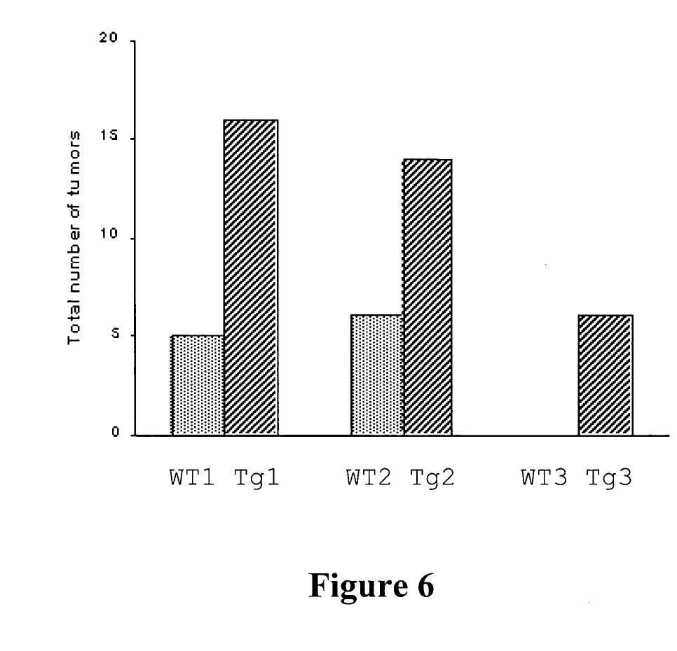

Intestinal tumors were induced in the Tie-CD66a-transgenic mice of Example 4 by using the carcinogen azoxymethan to show that the angiogenic action of CD66a supports the growth of tumors in vivo. 10 μg azoxymethan per 1 g body-weight of the mouse were injected once a week over a total of 8 weeks, the induced tumors were allowed to retain for further 8 weeks and the mice were sacrificed 16 weeks after the initial azoxymethane injection. In the transgenic mice, more and bigger tumors developed as compared to the wild type mice (FIG. 6). Considering the angiogenic action of CD66a as shown in the aortic explanation assay, this experiment clearly shows that the expression of CD66a in blood vessels leads to an increased tumor growth in vivo. Thus, agents that block the action of CD66a in endothelial cells will suppress tumor growth in vivo.

### EXAMPLE 8

### Inhibition of tumor growth in a SCID mouse model by an anti-murine CD66a antibody

In order to show that the functional inhibition of CD66a inhibits the growth of tumors in vivo, a tumor transplantation model has been developed. Since the monoclonal antibody 4D1/C2 binds to human, but not to murine CD66a, it could not be used in this in-vivo model. Instead, the monoclonal antibody AgB10 binding to murine CD66a was applied. This monoclonal antibody does not cross react with human CD66a.

To assess the therapeutic potential of CD66a-specific substances in their tumor-inhibitory qualities in vivo, a SCID mouse (severe combined immune deficient mice) model was used (Bosma, M. J. Immunodeficiency Reviews. 3:261-276, 1992). In these mice, a human adenocarcinoma cell line of the colon (HT29) was injected to establish solid tumors. The advantages of this model are (i) SCID mice do not reject xenogeneic tumors, (ii) SCID mice do not develop specific immune reactions adversing the effects of xenogenic antibodies and antigens and (iii) human carcinoma cell lines transplanted into the mice are nourished by murine blood vessels exclusively. Since the monoclonal antibody used for the inhibition of tumor growth does not bind to human CD66a, any anti-murine-CD66a-directed inhibition is targeting host tissues only.

SCID mice, aged 8-11 weeks, were injected HT29 (human colonic adenocarcinoma cell line) cells (1×10⁶ cells/mouse) subcutaneously between the scapulae. One day after injection of the tumor cell lines, inhibitory substances were injected intraperitoneally. Each mouse was either treated with anti-murine CD66a antibody AgB10 (described in Rudinskaya et al.: Biol. Membr. (USSR) 1987; (4):194-207 and Kuprina et al.; Histochemistry 1990; 94(2):179-86)((1 mg/injection), the corresponding IgG control antibody (1 mg/injection) or soluble recombinant murine CD66a (0.4 mg/injection).

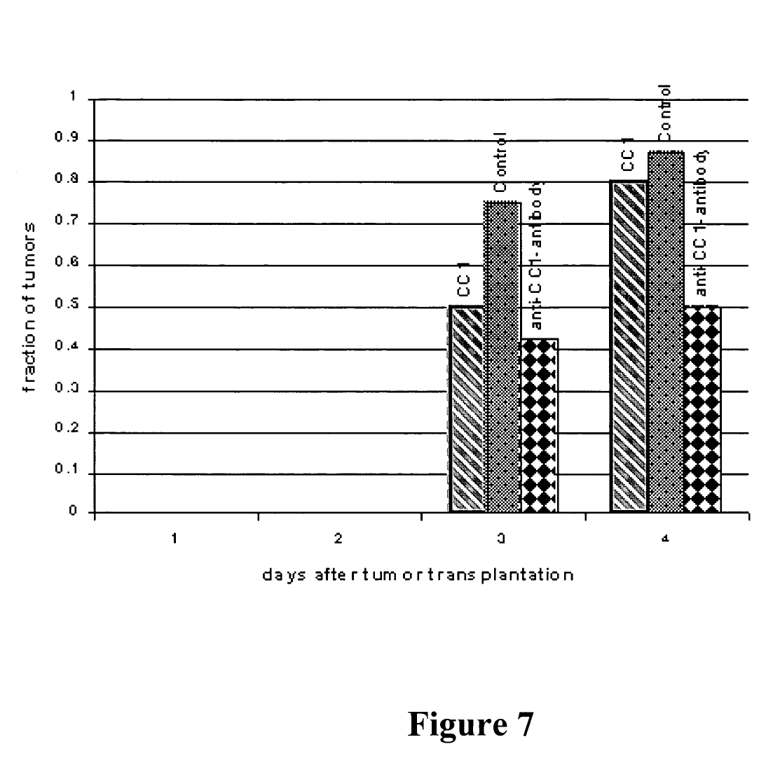

As shown in FIG. 7, at day 4 post transplantation, in only 5 out of 10 of the animals receiving the CD66a antibody tumors were observed after a single injection of antibody. In contrast, tumors were observed in 7 out of 8 of control animals (injection of non-immune IgG). (p<0.05). The injection of soluble recombinant CD66a did not affect tumor growth significantly.

That which is claimed is:

1. A method for influencing angiogenesis wherein (i) angiogenesis is improved by administering a therapeutically active amount of a compound in a pharmaceutically compatible carrier selected from the group consisting of CD66a, a CD66a variant, CD66a fragments or CD66a-derived glycostructures, or CD66a ligands, ligand fragments or structures derived therefrom, as well as substances inducing the expression of CD66a or CD66a ligand or (ii) angiogenesis is inhibited by administering a therapeutically active amount of a compound in a pharmaceutically compatible carrier selected from the group consisting of anti-CD66a specific antibodies and anti-sense oligonucleotides.

2. The method of claim 1 wherein angiogenesis is inhibited in tumor cells, comprising administering an antibody specific for CD66a in a pharmaceutically compatible carrier, wherein said antibody is effective for reducing formation of capillaries in the tumor cells by functionally blocking CD66a on tumor endothelial cell.

3. The method of claim 2, wherein the antibody binds to the B1 domain of CD66a.

4. The method of claim 2, wherein tumor angiogenesis of lung cancer, breast cancer, and colon carcinoma is reduced.

5. The method of claim 2, wherein the antibody is a monoclonal antibody.

6. The method of claim 5, wherein the antibody is the anti-CD66a antibody which was deposited with DMSZ (German-Type Collection of Microorganisms and Cell Cultures) Braunschweig under DSM ACC2371 on Oct. 22, 1998.

7. A sterile pharmaceutical composition for reducing angeniogenesis in tumor cells, comprising a monoclonal anti-CD66a antibody which was deposited with DMSZ (German-Type Collection of Microorganisms and Cell Cultures) Braunschweig under DSM ACC2371 on Oct. 22, 1998 and a pharmaceutically active carrier.

8. A method of inhibiting angiogenesis in tumor cells, comprising administering a sterile composition comprising an antibody specific for CD66a in a pharmaceutically compatible carrier, wherein said antibody is in an amount effective for reducing formation of capillaries in the tumor cells by functionally blocking CD66a on tumor endothelial cell and wherein the antibody is a monoclonal anti-CD66a antibody which was deposited with DMSZ (German-Type Collection of Microorganisms and Cell Cultures) Braunschweig under DSM ACC2371 on Oct. 22, 1998.
